(19) United States
(12) Patent Application Publication
(10) Pub. No.: US 2026/0132098 A1
(43) Pub. Date: May 14, 2026

(54) FLUORINATED NITRO COMPOUNDS HAVING A LOW GLOBAL WARMING POTENTIAL

(71) Applicant: SOLVAY SPECIALTY POLYMERS ITALY S.P.A., Bollate (Milano) (IT)

(72) Inventors: Emanuela Antenucci, Saronno (IT); Letanzio Bragante, DUE CARRARE (PD) (IT); Valeriy Kapelyushko, Alessandria (IT); Cristiano Monzani, Trezzo sull'Adda (IT); Vito Tortelli, Milano (IT); Francesco Venturini, Origgio (IT)

(73) Assignee: SOLVAY SPECIALTY POLYMERS ITALY S.P.A., Bollate (Milano) (IT)

(21) Appl. No.: 19/112,563

(22) PCT Filed: Sep. 16, 2022
(86) PCT No.: PCT/EP2022/075771

§ 371 Date: Mar. 18, 2025

(51) Int. Cl. C07C 205/03; C07C 205/02; H05K 7/20

(52) U.S. Cl. CPC C07C 205/03; C07C 205/02; H05K 7/20772

(57) ABSTRACT

The present invention relates to novel fluorinated nitrocompounds having low GWP and to the use of fluorinated nitrocompounds having low GWP as heat transfer fluids in various applications.

### TECHNICAL FIELD

The present invention relates to selected fluorinated nitro compounds having low GWP and to a method for exchanging heat with an object using compositions comprising selected fluorinated nitro compounds having low GWP as heat transfer fluids.

### BACKGROUND ART

Heat transfer fluids are known in the art for applications in heating and cooling systems; typically, heat transfer fluids include water, aqueous brines, alcohols, glycols, ammonia, hydrocarbons, ethers and various halogen derivatives of these materials, such as chlorofluorocarbons (CFCs), hydrochlorofluorocarbons (HFCs), (per)fluorinated polyethers (PFPEs) and the like.

Heat transfer fluids are used to transfer heat from one body to another, typically from a heat source to a heat sink so as to effect cooling of the heat source, heating of the heat sink or to remove unwanted heat generated by the heat source. The heat transfer fluid provides a thermal path between the heat source and the heat sink; it may be circulated through a loop system or other flow system to improve heat flow or it can be in direct contact with heat source and heat sink. Simpler systems use simply an airflow as heat transfer fluid, more complex system use specifically engineered gases or liquids which are heated or refrigerated in a portion of the system and then are delivered in thermal contact with the destination.

A preferred method of use of the heat transfer fluids of the invention is a method wherein the fluids are used to transfer heat with electrical or electronic equipment and in particular to remove excess heat generated by the functioning of the electrical or electronic equipment. This application has high industrial importance since electrical and electronic equipment typically has a relatively narrow range of temperatures within which their performance is optimal. These applications include, as it will be specifically detailed later, thermostatic baths and cooling circuits. Notably, the fluids of the invention are dielectric fluids so that they are electrical insulators and can transfer heat to and from electrical and electronic equipment while being directly in contact with the electronics such as wires and integrated circuits. Also, when these fluids are used in cooling circuits wherein they are circulated in closed pipes (as it will be described more in detail below) so that they are not supposed to contact directly the electrically conductive parts of the equipment, the use of dielectric fluids is particularly advantageous because it reduces the risk of damage to the electrical or electronic equipment in case of leaks.

Computing equipment such as computers, servers and the like generate substantial amounts of heat. Massive developments concentrating a large number of computers operating in shared locations such as server farms are getting more and more common. The industry of server farms, bitcoin mining farms and other supercomputing applications is growing extremely fast. A key factor in determining the building strategy of such installations is a control system which allows exchanging heat with such computing equipment. This system, often called “thermal management system”, is typically used for cooling the computing equipment during its operation, but it can also be used for heating e.g. when starting up a system in a cold environment. Air is still the most commonly used fluid which however has the drawback to require large air gaps between electronic boards, which causes the installation to have very large footprints. Air cooling also requires massive air conditioning engines and their energy consumption is extremely high and represents a significant portion of the running costs for such installations.

Recently, solutions for thermal management of servers based on the use of heat transfer fluids, especially liquid heat transfer fluids, are getting a lot interest because they are both energy efficient (use less energy than traditional air conditioning systems) and allow to pack more servers, processors and circuit boards in a smaller space.

Other important specialized applications for heat transfer fluids can be found e.g. in the semiconductor industry (TCUs, thermostatic baths, vapor phase soldering) and in the batteries industry especially in the vehicle battery industry for their thermal management systems.

A variety of heat transfer fluids exist which are used industrially in various applications, however the choice of an appropriate fluid can be critical in some cases. Several of the heat transfer fluids commonly used in the past are no longer viable because of their toxicity (ammonia, ethylene glycol), others have been phased out due to their environmental profile because they are not biodegradable and/or because they are considered to be detrimental to the earth ozone layer and/or to act as greenhouse gases if dispersed in the environment.

Fluorinated liquids are very effective heat transfer fluids. Commercial products exist such as Solvay's Galden® PFPE and 3M's Fluorinert™: these are liquid polymers or oligomers which are dielectric, have a high heat capacity, a low viscosity and are non-toxic and chemically inert so they can get in direct contact with electronic boards and also do not chemically interact with most materials. A drawback associated with these fluorinated fluids used so far is their high GWP value.

GWP (Global Warming Potential) is an attribute which can be determined for a given chemical compound which indicates how much heat a given greenhouse gas can entrap in the atmosphere (considering “1” as the reference value for CO₂) and is calculated over a specific interval of time, typically 100 years (GWP₁₀₀).

The determination of GWP₁₀₀ is performed by combining experimental data concerning the atmospheric lifetime of the chemical compound and its radiative efficiency with specific computational tool which are standard in the art and are described e.g. in the extensive review published by Hodnebrog et. Al. in Review of Geophysics, 51/2013, p 300-378. Highly stable halogenated molecules such as CF₄ and chloro/fluoro alkanes have a very high GWP₁₀₀ (7350 for CF₄, 4500 for CFC-11).

Over the years heat transfer fluids having elevated values of GWP have been phased out by the industry and replaced with compounds having a lower GWP₁₀₀ value and there is still a continuous interest in heat transfer fluids having GWP₁₀₀ values which are as low as possible.

Hydrofluoroethers, in particular segregated hydrofluoroethers, tend to have relatively low GWP₁₀₀ values while the rest of their properties can be compared to those of the CFCs used in the past, for this reason some hydrofluoroethers have been used industrially and gained popularity as heat transfer fluids and are marketed e.g. by 3M under the trade name “Novec®”.

Hydrofluoroethers are broadly described as heat transfer media due to their wide temperature range where they are liquid, and due to their low viscosity in a broad range of temperatures which makes them useful for applications as low temperature secondary refrigerants for use in secondary loop refrigeration systems where viscosity should not be too high at operating temperatures.

Fluorinated ethers are described for example by 3M in U.S. Pat. No. 5,713,211, by Dupont in US 2007/0187639 and by Solvay Solexis S.p.A in WO 2007/099055 and WO2010/034698.

However, while much lower than CFCs, the GWP₁₀₀ of segregated hydrofluoroethers is still in a range from 70 to 500 as shown in U.S. Pat. No. 5,713,211 (table 5):

| | GWP₁₀₀ |
|---|---|
| C₄F₉—O—CH₃ | 330 |
| C₄F₉—O—C₂H₅ | 70 |
| c-C₆F₁₁—O—CH₃ | 170 |

Other hydrofluoro-olefins have been commercialized as heat transfer fluids e.g. by Chemours (Opteon™). These compounds have a very low GWP, around 1, but, differently from the formerly cited compounds, are much more flammable and therefore this limits their field of use.

International PCT patent application EP2020/057121 to Solvay Specialty Polymers Italy S.p.A describes in general the use of halogenated vinyl ethers as heat transfer fluids having low GWP, low flammability and good dielectrical properties and thermal capacity. Still there is a continued need to provide even more effective methods for effective heat transfer using heat transfer fluids which have an even better balance of good thermal and dielectric properties, are liquid in a broad range of temperatures, are non flammable, and have very low GWP₁₀₀ (30 and below).

### SUMMARY OF INVENTION

In one aspect the present invention relates to fluorinated nitrocompounds having a general formula selected from (I) and (II):

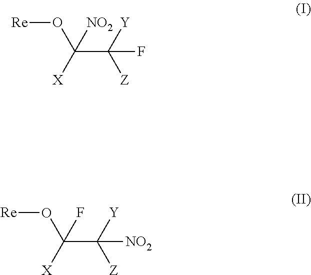

wherein
   (i) X, Y and Z are independently selected from:
      halogen,
      hydrogen,
      partially or fully halogenated C₁-C₄ alkyl,
with the proviso that at least one of X, Y or Z is a halogen, and
   (ii) Re is selected from any C₁-C₇ linear, branched, saturated or unsaturated, or C₅-C₇ cyclic, saturated or unsaturated carbon chain which can be, optionally, partially or fully halogenated, and can comprise 0-5 heteroatoms selected from O and S, with the proviso that if Re is CF₃ at least one of X, Y and Z is not F.

In another aspect, the present invention relates to a method for exchanging heat with an object said method comprising using a heat transfer fluid wherein said heat transfer fluid comprises one or more fluorinated nitrocompounds having a general formula selected from (I) and (II):

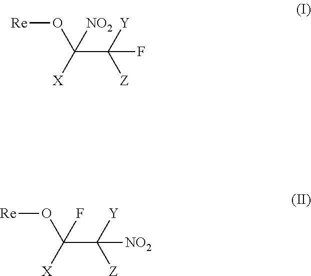

wherein
   (i) X, Y and Z are independently selected from:
      halogen,
      hydrogen,
      partially or fully halogenated C₁-C₄ alkyl,
with the proviso that at least one of X, Y or Z is a halogen, and
   (ii) Re is selected from any C₁-C₇ linear, branched, saturated or unsaturated, or C₅-C₇ cyclic, saturated or unsaturated carbon chain which can be, optionally, partially or fully halogenated, and can comprise 0-5 heteroatoms selected from O and S.

### DESCRIPTION OF EMBODIMENTS

For “electronic computing equipment” it is intended any individual or arrays of individual computer boards comprising microprocessors CPUs, GPUs, SSD and DDR Memory, and performing computational work, thus including both large server farms, internet servers, bitcoin mining factories, but also smaller individual computers, internet servers, computer gaming equipment. Both large and small installation may benefit from the heat transfer method of the present invention.

The term “semiconductor device” in the present invention includes any electronic device which exploits the properties of semiconductor materials. Semiconductor devices are manufactured both as single devices and as integrated circuits which consist of a number (which can go from two to billions) of devices manufactured and interconnected on a single semiconductor substrate or “wafer”. The term “semiconductor devices” includes both the basic building blocks, such as diodes and transistors, to the complex architectures built from these basic blocks which extend to analog, digital and mixed signal circuits, such as processors, memory chips, integrated circuits, circuit boards, photo and solar cells, sensors and the like. The term “semiconductor devices” also includes any intermediate or unfinished product of the semiconductor industry derived from a semiconductor material wafer.

In one aspect the present invention relates to fluorinated nitrocompounds having a general formula selected from (I) and (II) below:

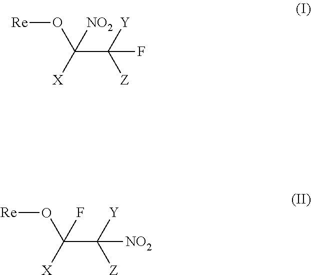

wherein:
   (i) X, Y and Z are independently selected from:
      halogen,
      hydrogen,
      partially or fully halogenated C₁-C₄ alkyl,
with the proviso that at least one of X, Y or Z is a halogen, and
   (ii) Re is selected from any C₁-C₇ linear, branched, saturated or unsaturated, or C₅-C₇ cyclic, saturated or unsaturated carbon chain which can be, optionally, partially or fully halogenated, and can comprise 0-5 heteroatoms selected from O and S, with the proviso that if Re is CF₃ at least one of X, Y and Z is not F.

Preferably, said at least one halogen in position X, Y and/or Z is independently selected from chlorine (CI) and fluorine (F), more preferably at least one of X, Y or Z is F.

Preferably, if one of more of X, Y and Z are partially or fully halogenated C₁-C₄ alkyl said halogen comprises, or more preferably is, F.

In a particularly preferred embodiment the fluorinated nitrocompounds of the invention are compounds according to the general formulae above wherein one of X, Y and Z, is “—CF₃”, one of X, Y and Z, is F and the remaining one of X, Y and Z is selected from Cl and F.

In another particularly preferred embodiment the fluorinated nitrocompounds of the invention are compounds according to the general formulae above wherein one of X, Y and Z, is F, and the remaining two of X, Y and Z are selected from Cl and F.

As mentioned above Re can be any C₁-C₇ linear, branched, saturated or unsaturated, or C₅-C₇ cyclic saturated or unsaturated carbon chain which can be, optionally, partially or fully halogenated, and can comprise 0-5 heteroatoms selected from O and S, with the proviso that if Re is CF₃ at least one of X, Y and Z is not F. In a further preferred embodiment the halogens in Re are selected from Cl and F and most preferably are all F. Preferably, if Re comprises one or more S atoms these are part of a sulfonic group selected from —SO₂F, —SO₃H, or —SO₃Me wherein Me is an alkali metal. Also preferably if Re comprises one or more oxygen atoms these are all either part of a sulfonic group as defined above or are engaged in ether bonds C—O—C.

In a particularly preferred embodiment “Re-” corresponds to the general formula:

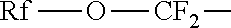

wherein:
   Rf can be any C₁-C₆ linear, branched, saturated or unsaturated, or C₅-C₆ cyclic saturated or unsaturated carbon chain which can be partially or fully halogenated, and can comprise 0-5 heteroatoms selected from O and S. Preferably said halogens in Rf are selected from Cl and F and are preferably all F. Preferably, if Rf comprises one or more S atoms these are part of a sulfonic group selected from —SO₂F, —SO₃H, or —SO₃Me wherein Me is an alkali metal. Also preferably if Rf comprises one or more oxygen atoms these are all either part of a sulfonic group as defined above or are engaged in ether bonds C—O—C.

Compounds in this class can be synthetized starting from common fluoroolefins such as fluorinated vinyl ethers and polyethers, via nitrofluorination reaction as it will described in more details below. When an olefin comprising a C═C double bond is subject to nitrofluorination the double bond is saturated, one —NO₂ radical is added to one of the carbon atoms forming the double bond, and an —F radical is added to the other.

The nitro group can bond to either of the two carbon atoms forming the double bond. As a result nitrofluorination of a non symmetric olefin may result in a mixture of 2 different isomers each one according to general formula (I) or (II), respectively. Within the mixture, one of the two compounds may be present in higher amount than the other. Without being bound by theory it is believed that both polarity of the double bond and steric effects play a role in the selectivity of the reaction. It is difficult to predict how specific the nitrofluorination reaction will be, but in general it has been observed that the —NO₂ group tends to preferentially bind to the carbon atoms which does not have an oxygen atom in alpha position and thus preferentially form compounds according to formula (II). In some cases, as it will be shown in the experimental section, the nitrofluorination of certain olefins was very specific so that only a compound according to formula (II) was formed.

Nitrofluorination of olefins is a known process and is described e.g. in Knunyants, I. L., German, L. S., & Rozhkov, I. N. (1963). “Aliphatic fluoro nitro compounds Communication 1. Conjugated nitrofluorination of olefins.” Bulletin of the Academy of Sciences, USSR Division of Chemical Science, 12(11), 1794-1797. https://doi.org/10.1007/BF00843794. However nitrofluorination reactions leading to the preparation of the nitrocompounds of the present invention have not been described.

Nitrofluorination of fluorinated compounds can be performed as generally known for other olefins. Preferably the nitrofluorination reaction for preparing the fluorinated olefins of the present invention is conducted in two steps:
1—Preparation of nitro/fluoro mixture and
2—Reaction of the halogenated olefin with the nitro/fluoro mixture

### Step 1. Preparation of Nitro/Fluoro Mixture

A nitro/fluoro mixture for the present invention can be prepared using several different methods, including but not limited to:
a—mixing pure nitric acid and anhydrous hydrogen fluoride in a molar ratio equal or higher than 1:1 (HF/HNO₃). (preferred range of molar ratio is 5:1-10:1)
b—reacting carbonyl-difluoride and nitric acid to form the nitro/fluoro mixture like in method “a”.
   Step 2—Reaction of the Halogenated Olefin with the Nitro/Fluoro Mixture

The halogenated olefin is slowly added to the nitro/fluoro mixture placed in a metal reactor in a stoichiometric or slightly overstoichiometric ratio. Internal temperature is carefully monitored. The reaction proceeds while the reactor is maintained at a temperature in the range of 20-120° C. for a time between 3 and 24 hours. The choice of temperature depends on the selected olefin and on its reactivity and stability. In general below 20° C. reaction kinetic is too slow and over 120° C. certain olefins can be unstable. At the end of the reaction the reactor is unloaded and the crude product is separated and purified.

The nitrofluorination reaction can be carried out in a pressurized reactor, in an atmospheric stirred tank reactor

The described nitrofluorination methods should not be considered as limiting. A skilled person would be able to apply variation and changes to the methods, and in particular to scale up the method for industrial application following conventional chemical engineering procedures.

The present invention also relates to a method for exchanging heat with an object said method comprising using a heat transfer fluid wherein said heat transfer fluid comprises one or more chemical compounds having a general formula selected from (I) and (II) below:

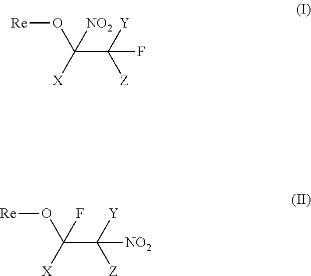

wherein
   (i) X, Y and Z are independently selected from:
      halogen,
      hydrogen,
      partially or fully halogenated C₁-C₄ alkyl,
with the proviso that at least one of X, Y or Z is a halogen, and
   (ii) Re is selected from any C₁-C₇ linear, branched, saturated or unsaturated, or C₅-C₇ cyclic, saturated or unsaturated carbon chain which can be, optionally, partially or fully halogenated, and can comprise 0-5 heteroatoms selected from O and S.

Preferably, said at least one halogen in position X, Y and/or Z is independently selected from chlorine (CI) and fluorine (F), more preferably at least one of X, Y or Z is F.

Preferably, if one of more of X, Y and Z are partially or fully halogenated C₁-C₄ alkyl said halogen comprises, or more preferably is, F.

In a particularly preferred embodiment the fluorinated nitrocompounds of the invention are compounds according to the general formulae above wherein one of X, Y and Z, is “—CF₃”, one of X, Y and Z, is F and the remaining one of X, Y and Z is selected from Cl and F.

In another particularly preferred embodiment the fluorinated nitrocompounds of the invention are compounds according to the general formulae above wherein one of X, Y and Z, is F, and the remaining two of X, Y and Z are selected from Cl and F.

As mentioned above Re can be any C₁-C₇ linear, branched, saturated or unsaturated, or C₅-C₇ cyclic saturated or unsaturated carbon chain which can be, optionally, partially or fully halogenated, and can comprise 0-5 heteroatoms selected from O and S. In a further preferred embodiment the halogens in Re are selected from Cl and F and most preferably are all F. Preferably, if Re comprises one or more S atoms these are part of a sulfonic group selected from —SO₂F, —SO₃H, or —SO₃Me wherein Me is an alkali metal. Also preferably if Re comprises one or more oxygen atoms these are all either part of a sulfonic group as defined above or are engaged in ether bonds C—O—C.

In a particularly preferred embodiment “Re-” corresponds to the general formula:

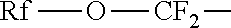

wherein:
   Rf can be any C₁-C₆ linear, branched, saturated or unsaturated, or C₅-C₆ cyclic saturated or unsaturated carbon chain which can be partially or fully halogenated, and can comprise 0-5 heteroatoms selected from O and S. Preferably said halogens in Rf are selected from Cl and F and are preferably all F. Preferably, if Rf comprises one or more S atoms these are part of a sulfonic group selected from —SO₂F, —SO₃H, or —SO₃Me wherein Me is an alkali metal. Also preferably if Rf comprises one or more oxygen atoms these are all either part of a sulfonic group as defined above or are engaged in ether bonds C—O—C.

The method of the invention can be used to exchange heat with any object. The method of the invention is particularly useful when the object is an electronic computing equipment such as e.g. computer servers. In fact the method of the present invention employs heat transfer fluids which are stable, dielectric, non flammable and non corrosive so that it can also be used in systems using the so called “immersion cooling” or “direct contact cooling”. In these systems the fluids are placed in direct contact with the electronic circuit boards. Such fluids at their working temperature can be gaseous, liquids (single phase immersion cooling) or be in a gas/liquid equilibrium (i.e. around the boiling point of the liquid, in the so called “two phase immersion cooling”).

In immersion cooling, electronic computer equipment such as CPUs, GPUs, Memory, and other electronics, including complete servers, are completely immersed in a thermally conductive dielectric liquid or coolant, which temperature is controlled through the use of a circulation system which pumps the liquid trough pipes and to heat exchangers or to radiator type coolers to reject the heat from the coolant.

Server immersion cooling is becoming a popular solution for server cooling solution, as it allows to drastically reduce energy usage through the elimination of the expensive air conditioning infrastructure. These systems are replaced with efficient low speed liquid circulation pumps and simpler heat exchanger and/or radiator systems.

The temperatures used in liquid immersion cooling are determined by the highest temperature at which the devices being immersed can reliably operate. For servers this temperature range is typically between 15 to 65° C., however in some cases this range is extended up to 75° C.

Current commercial applications for immersion cooling range from data center oriented solutions for commodity server cooling, server clusters, HPCC applications and Cryptocurrency Mining and mainstream cloud-based and web hosting architectures. Liquid immersion cooling is also used in the thermal management of computing equipment related to LEDs, Lasers, X-Ray machines, and Magnetic Resonance Imaging devices.

The method of the present invention is suitable for both single phase immersion cooling and two phase immersion cooling. In a single-phase immersion cooling system, servers are immersed is a bath of dielectric fluid. Heat is transferred to the fluid via direct contact with the electronic components. The fluid is maintained at constant temperature usually through a recirculation system typical of thermostatic baths. A more or less sophisticated control system may be present controlling the instant temperature of the fluid and the temperature of the servers optimizing the fluid temperature in each moment.

Two phase immersion cooling is a technology marketed by Allied Control and a few other players, which involve submerging the electronic devices in closed tanks with a bath of dielectric fluid where the dielectric fluid has a boiling point corresponding to a desired temperature at which the bath can be set. When the electronic devices heat up to the boiling point of the dielectric fluid the fluid turns into vapor subtracting heat. The vapor then condenses on a lid or coil condenser placed above the bath and precipitates again in the bath. A design of this type is particularly appreciated because it does not require recirculation of the dielectric fluid, and allows packing more electronic devices is a small space.

Heat transfer fluids according to the invention i.e. comprising one or more compounds according to formulae (I) or (II) as defined above can be formulated with compounds having boiling points between 40° C. and 90° C. (see examples in the experimental section below) and therefore find particular application in dual phase immersion cooling.

Still in the field of computer equipment cooling, the method of the invention can also be used in non immersion cooling system where the heat transfer fluid is circulated in a closed system and brought in thermal contact with the processors trough plates of thermally conductive materials, such as the server cooling solutions produced by Ebullient under the name of “module loops”. The use of a dielectric fluid such as the heat transfer fluid of the present invention is anyway beneficial because the risk of leakages is always present and conductive liquids may have destructive effects on the electronics.

The method of the invention can also find application for example in the semiconductor industry where temperature control during manufacturing of semiconductor devices is of great importance. In this case the object which exchanges heat with the heat transfer fluid of the invention is a semiconductor device. Temperature control units (TCUs) are used all along the production line for the fabrication of semiconductor devices, and heat transfer fluids according to the invention can be employed to remove unwanted heat during steps like wafer etching and deposition processes, ion implantation and lithographic processes. The heat transfer fluid is typically circulated through the wafer mounts and each process tool which requires temperature control has its own individual TCU.

Some tools of particular importance which include TCUs are silicon wafer etchers, steppers and ashers. Etching is performed using reactive plasma at temperatures ranging from 70° C. to 150° C. and the temperature of the wafer must be controlled precisely with the entire temperature range during the plasma treatment. Following the plasma treatment the etched parts are normally immersed in a solvent which removes the etched parts. This second step does not normally require temperature control as it is performed at mild or ambient temperature. When referring to an “etcher” in the present application, it is intended the equipment wherein the plasma treatment at high temperature is performed and which therefore requires a TCU.

Steppers are used in the photolithography of wafers to form the reticules which are then used to expose the photosensitive mask. This process is carried out at temperatures between 40° C. and 80° C., however temperature control is extremely important as the wafer need to be maintained at a precise fixed temperature (+/−0.2° C.) along the process to ensure good results.

Ashing is a process where the photosensitive mask is removed from the wafer and is performed at temperatures from 40° C. to 150° C. The system uses plasma and also here precise temperature control is particularly important.

Another relevant process is plasma enhanced chemical vapour deposition (PECVD) wherein films of silicon oxide, silicon carbide and/or silicon nitride are grown on a wafer within a chamber. Also in this case, while the temperature at which this step is performed can be selected in the range between 50° C. and 150° C., during the deposition process the wafer must be kept uniformly at the selected temperature.

In a semiconductor device production facility typically each Etcher, Asher, Stepper and plasma enhanced chemical vapor deposition (PECVD) chamber has its own TCU wherein, applying the method of the invention, a heat transfer fluid according to the invention can be recirculated.

Another process step where heat transfer fluids of the invention are used in the semiconductor industry is vapor phase reflow (VPR) soldering. This is the most common method used to connect surface mount devices, multi chip modules and hybrid components to circuit boards. In this method the soldering material is applied in paste form and then the semiconductor device e.g. an unfinished circuit board is placed in a closed chamber with heat transfer fluid at its boiling point in equilibrium with its vapor phase. The fluid in vapor phase transfers heat to the soldering paste which then melts and stabilize the contacts. In this case the fluid is in direct contact with the circuit board so that it must be dielectric and non corrosive. For this application is important that the heat transfer fluid comprises compounds having a boiling point which is sufficient to melt the soldering paste. Also in this process heat transfer fluids according to the present invention can be used.

Another system which is a key part of the production process of many semiconductor devices is thermal shock testing. In thermal shock testing a semiconductor device is tested at two very different temperatures. Different standards exist, but in general the test consists in subjecting the semiconductor device to high and low temperatures and then testing the physical and electronic properties of the device. Typically the semiconductor device to be tested is directly immersed alternatively in a hot bath (which can be at a temperature of from 60° C. to 250° C.) and a cold bath (which can be typically at a temperature of from −10° C. to −100° C.). The transfer time between the two baths must be minimized, generally below 10 seconds. Also in this test the fluid making up the baths goes in direct contact with the device and therefore must be dielectric and non corrosive. In addition, to avoid contamination of the baths, it is highly preferable that the same fluid is used both in the cold and in the hot bath. Therefore heat transfer fluids which exist as liquids in a broad range of temperatures are preferred. Also in this case heat transfer fluids according to the invention can be used in this process.

Heat transfer fluids for use in the manufacturing of semiconductor devices are typically liquids which are dielectric, non corrosive, and exist in the liquid state in a broad range of temperatures with relatively low viscosity which makes them easily pumpable.

The method of the invention can be used in all the steps of the manufacturing of semiconductor devices which require the semiconductor device to exchange heat with a heat transfer fluid. In particular when using semiconductor processing equipment such as an Etcher, an Asher, a Stepper and a plasma enhanced chemical vapor deposition (PECVD) chamber: each of these equipment requires precise temperature control and/or heat dissipation and therefore they include temperature control units (TCUs) which can include the selected heat transfer fluid of the method of the invention.

Additionally, in thermal shock testing, which is an integral part of semiconductor device manufacturing because only those devices which pass the test are processed further, the semiconductor device is cooled and heated using at least two baths made of heat transfer fluids, a cold one typically at a temperature of from −10 to −100° C., and a hot one typically at a temperature of from 60° C. to 250° C. The method of the invention can be advantageously performed selecting the appropriate compound or blend of compounds according to the general formulae (I) and/or (II) for making up the heat transfer fluid for the baths. Preferably the heat transfer fluid should be selected so that the same heat transfer fluid can be used in both bath thanks to the large temperature range in which the fluid is in liquid state, so that there is no risk of cross contamination of the baths.

The method of the invention can also find application in vapor phase soldering, in fact the selected heat transfer fluid of the method of the invention can be formulated so to have a boiling point in line with that of the soldering paste, so that a semiconductor device comprising soldering paste which still has to be “cured” can be introduced into a closed chamber which contains the selected heat transfer fluid of the method of the invention at its boiling point in equilibrium with its heated vapors. The heated vapors will transfer heat to the semiconductor device thereby melting the soldering paste and therefore fixing the contacts as needed. For this application high boiling point compounds according to the general formulae will need to be used in the heat transfer fluid.

An additional advantage is that a single heat transfer fluid can be used in multiple applications potentially allowing the use of a single heat transfer fluid across an entire semiconductor devices manufacturing facility.

Another area wherein the method of the present invention can find application is the thermal management of batteries, in particular rechargeable batteries such as vehicle batteries for cars, trams, trains and the like.

Currently, most of the development in the field of rechargeable batteries, in all industry segments, is focused on Lithium-ion based batteries which are based on different types of lithium salts. Batteries based on Lithium Manganese Oxide, Lithium Iron Phosphate and Lithium Nickel Manganese Cobalt Oxide find application e.g. in vehicles, power tools, e-bikes, and the like. Batteries based on Lithium Cobalt Oxide are typically used in smaller sizes and less intensive applications such as cell phones, portable computers and cameras. Batteries based on Lithium Nickel Cobalt Aluminum Oxide and Lithium Titanate are being considered in applications requiring high power and/or capacity such as electric powertrain and grid storage. Naturally also new technologies, outside the realm of Lithium-ion based batteries, are being explored and continuously developed. The method of the present invention is not linked to a particular battery technology and is applicable to both current and future generations of battery systems.

Differently from conventional power systems, batteries, and in particular rechargeable batteries, have strict requirements for their working environment. Batteries tend to operate in the best conditions within a relatively narrow range of temperatures.

In general, low temperatures have an effect on the battery chemistry slowing down the reaction rate and therefore reducing the electricity flow when charging or discharging. High temperatures instead increase the reaction rate and at the same time also increase energy dissipation thus generating even more excess heat possibly causing an uncontrolled increase of temperature which can cause irreversible damage to the cell. For a typical Li-ion battery a temperature above 80° C., even only in a part of its structure, can start exothermal chemical reactions which cause a further temperature increase of the battery, ultimately leading to a complete collapse of the battery with risk of fire and explosion.

On the other hand, the practical applications of batteries require them to be efficient in a much broader range of temperatures. For example vehicles batteries need to function properly in any environment where people is expected to use them, so that they need to be operative in a temperature range from −20° C. to +40° C. and beyond. In addition to that, charge and discharge cycles of batteries can generate heat within the battery itself making it even more difficult to maintain the battery within an acceptable temperature range.

Indicative figures for a typical Li-ion battery suggest that the usable range is normally from −20° C. to 60° C., but a good power output is only obtained from 0° C. to 40° C., where optimal performance is only obtainable from 20° to 40° C. Temperature also affects battery duration, in fact the number of charge/discharge cycles a battery can withstand before being considered exhaust go down quickly below 10° C. due to anode plating, and above 60° C. due to the deterioration of the electrode materials. The temperature ranges for optimal performance may be different for different battery chemistries and constructions, however all current commercial batteries share a relatively narrow temperature window where their performance is optimal. It is also important in general to ensure that the entire battery is kept uniformly at the same temperature without hot or cold zones, as this can reduce its lifetime and safety.

For this reason it is nowadays standard to integrate a Battery Thermal Management System (BTMS) within commercial battery assemblies, especially when safety, reliability and lifetime of the battery are a significant concern. These BTMSs can be more or less complex, depending on the type of battery, however one common element is the presence of a heat transfer fluid such as a gas or a liquid which exchanges heat with the battery thus heating or cooling it.

Battery thermal management systems (BTMSs) are therefore extremely important, especially in applications requiring high power, and high reliability such as vehicles batteries. A BTMS can be more or less complex, depending on the application, but each BTMS has at least a function to cool the battery when its temperature is too high and a function to heat the battery when its temperature is too low, typically using a heat transfer fluid which exchanges heat with the battery. Other common features in BTMSs are an insulation system, to reduce the effect of the external environment on the battery temperature, and a ventilation system which helps dissipating hazardous gases which may develop within the battery pack. However the method of the present invention relates specifically to the heat exchange function of a BTMS and can be applied easily to any BTMS and integrated with its other functions and features.

BTMSs using a liquid as heat transfer fluid are common because liquids can transfer a larger amount of heat more quickly than gases. Typically the fluid is circulated by a pump within a closed system which is in thermal contact with the battery and with a second system which has the function of heating and/or cooling the fluid to the desired temperature. This second system may comprise any combination of a refrigeration system and a heating system or may combine heating and cooling functions in a heat pump. The circulating fluid absorbs heat from or release heat to the battery and then it is circulated in said second system to bring the fluid back to the desired temperature. A more or less sophisticated control system may be present controlling the instant temperature of the fluid and the temperature of the battery optimizing the fluid temperature in each moment.

In some systems the heat transfer fluid can go in direct contact with the battery cells which are then immersed in it. Clearly in these cases the fluid must be dielectric in order to protect the battery cells and their electronic components. In other cases the heat transfer fluid is circulated in a separate closed system which only exchanges heat by indirect contact trough e.g. a heat exchange plate made of metal or other thermally conductive material. A dielectric fluid may be beneficial also in this type of systems because closed systems have anyway a high risk of leakages.

Particularly for high power batteries thermal management systems based on fluids, liquids in particular, are being used and the method of the invention offers an improved thermal management at a lowered GWP₁₀₀.

Heat transfer fluids according to the invention can find application as heat transfer fluids in BTMS.

Beyond the cited applications, the method of the invention can be also adapted to any heat exchange method e.g. for heating or cooling compartments (e.g. food stuffs compartments) including those on board of aircrafts, vehicles or boats, for heating or cooling industrial production equipment, for heating or cooling batteries during their operations, for forming thermostatic baths.

As mentioned in the introduction, heat transfer fluids used in these fields include fluorocompounds. In particular hydrofluorotethers have found application in these fields due to their chemical inertness, dielectricity, wide range of T in which they are liquid and pumpable (typically having a viscosity between 1 and 50 cps at the temperatures of use), low flammability and relatively low GWP.

Commercially available hydrofluoroethers for use in these fields are e.g. those from the Novec™ series of 3M which combine all these properties with a relatively low GWP₁₀₀ of from about 70 to 300.

Still, GWP is a critical property nowadays also due to the regulatory environment so that there is always a demand to develop new heat exchange fluids which have even lower GWP than then currently commercialized hydrofluoroethers.

The applicant has surprisingly found that the heat transfer fluid employed in the method of the invention is non-flammable, provides efficient heat transfer, can be used across a wide temperature range and has equal or improved dielectric and thermal properties with respect to other materials commercialized as heat transfer fluids. Surprisingly heat transfer fluids used in the invention have an extremely low GWP₁₀₀, in general lower than 30 and for some materials even lower than 3, as it will be shown below in the experimental section. This is a particularly unexpected result and in fact previous reviews such as Hodnebrog et. al. cited above did not investigate or propose fluorinated compounds having nitro groups as low GWP compounds.

Therefore, using these selected chemical compounds in accordance to the general formulae (I) and/or (II) heat transfer fluids can be formulated which have a GWP₁₀₀ value of less than 30, preferably less than 10, even more preferably less than 5. The heat transfer fluids according to the invention also have low toxicity, showing good heat transfer properties and relatively low viscosity across the whole range. Also, the fluids of the invention have good electrical compatibility, i.e. they are non corrosive, have high dielectric strength, high volume resistivity and low solvency for polar material. The electrical properties of the fluids of the invention are such that they can be used in immersion cooling system for electronics in direct contact with the circuits as well as in indirect contact applications using loops and/or conductive plates.

The heat transfer fluids for use in the method of invention preferably comprise more than 5% by weight of one or more compounds according to formulae (I) and (II) as defined above, more preferably more than 50% by weight, even more preferably more than 90% by weight. In one embodiment the heat transfer fluid is entirely made of one or more compounds according to the general formulae (I) and (II).

In some embodiments the heat transfer fluid of the invention comprises a blend of chemical compounds according to the general formulae.

In another aspect the present invention also encompasses an apparatus comprising an electronic computing equipment and a heat transfer fluid wherein said heat transfer fluid comprises one or more fluorinated nitrocompounds having a general formula selected from (I) and (II) as defined above.

In a further aspect the present invention relates to an apparatus comprising a battery, preferably a rechargeable battery, a thermal management system for said battery, said thermal management system for said battery comprising a heat transfer fluid exchanging heat with said battery, wherein said heat transfer fluid comprises one or more fluorinated nitrocompounds having a general formula selected from (I) and (II) as defined above.

Should the disclosure of any patents, patent applications, and publications which are incorporated herein by reference conflict with the description of the present application to the extent that it may render a term unclear, the present description shall take precedence.

The invention will be now described in more detail with reference to the following examples whose purpose is merely illustrative and not limitative of the scope of the invention.

### Standards:

Measurement of electrical properties were performed according to the following standards:
Dielectric constant—ASTM D924-15
SPECIFIC Heat Capacity—ASTM E1269

### EXAMPLES

The following fluoro-olefins were subject to nitrofluorination:
PVE (1,1,1,2,2,3,3-heptafluoro-3-[(1,2,2-trifluoroethenyl)oxy]propane))
CVE ((1,2-dichloro-1-fluoro-2-(trifluoromethoxy)ethene))
MOVE3 {difluoro[(1,2,2-trifluoroethenyl)oxy]methoxy}trifluoromethane

The nitrofluorination reaction was performed in a stainless steel reactor (0.6 L internal volume) which was loaded with 6.7 mol of AHF (Anhydrous Hydrogen Fluoride) and with 0.73 mol of WFNA (White Fuming Nitric Acid >95%) transferred under vacuum. The content was stirred and the temperature was raised to the temperature indicated in the table below.

A steel cylinder containing 0.6 mol of olefin was then connected to the reactor with a metal joint and a valve. The connection valve was then open allowing the olefin to go into the reactor.

The system was maintained at constant temperature for the time indicated in the table below then the reactor was cooled to room temperature. The content of the reactor was then transferred into a mixture of water and ice (1180 g) in a plastic washing bottle.

An organic phase was then separated from the water/ice mixture and washed twice with 10% Potassium hydroxide solution using a plastic separator funnel. Subsequently anhydrification on anhydrous Magnesium Sulfate was performed.

Finally, after filtering to remove Magnesium sulfate residues, the organic phase was distilled in a 30 cm, $14 mm, Hempel column with Raschig rings equipped with a reflux condenser.

The resulting material was analyzed by 19F-NMR spectrometry. As it can be seen form Table 1 below, the nitrofluorination of PVE and MOVE3 resulted in a mixture of two isomers according respectively to general formula (I) and (II) wherein the molar ratio is 1 to 3. The isomer according to formula (II) is present in larger amount. In the case of CVE its nitrofluorination was more selective as only the isomer according to general formula (II) was found.

When both isomers were present (according respectively to formula (I) and (II)) the purified organic phase was a mixture of the two isomers (distillation in these condition does not separate the isomers due to their boiling point being not very different)

Table 1 summarized the reaction condition and the products obtained:

TABLE 1

| | Reaction | | | Product |
|---|---|---|---|---|
| | Temperature | | | Boiling |
| Olefin | and time | Yield | Products | Point. |
| PVE | 60° C./24 h | 54% | [n-NFE-41-11] (according | 74-76° C. |
| | | | to general formula (II) and | |
| | | | [i-NFE-41-11] (according | |
| | | | to general formula (I)) | |
| | | | 3:1 molar ratio | |
| CVE | 35° C./3 h | 90% | n-NCFE-31-5 (according to | 101- |
| | | | general formula (II)) | 104° C. |
| MOVE3 | 60° C./24 h | 53% | [n-NFE-31-9] (according | N.D. |
| | | | to general formula (II) and | |
| | | | [i-NFE-31-9]. (according | |
| | | | to general formula (I) | |
| | | | 3:1 molar ratio | |

Chemical names of nitrofluorination products:

### PVE:

[n-NFE-41-11]1,1,1,2,2,3,3-heptafluoro-3-(1,1,2,2-tetrafluoro-2-nitroethoxy)propane
[i-NFE-41-11]1,1,1,2,2,3,3-heptafluoro-3-(1,2,2,2-tetrafluoro-1-nitroethoxy)propane

### CVE:

[n-NCFE-31-5](racemic mix) 1,2-dichloro-1,2-difluoro-1-nitro-2-(trifluoromethoxy)ethane

### MOVE3:

[n-NFE-31-9][difluoro(1,1,2,2-tetrafluoro-2-nitroethoxy) methoxy]trifluoromethane
[i-NFE-31-9][difluoro(1,2,2,2-tetrafluoro-1-nitroethoxy) methoxy]trifluoromethane

The nitrocompounds obtained were tested for their flammability (all were found to be not flammable, not combustible).

The compounds of the invention have surprisingly good specific heat capacity which ensures a more efficient heat transfer. This is combined with other desirable properties in line with the other materials (non flammability, low GWP and very low dielectric constant). These properties in combination make the materials of the invention particularly suitable as heat transfer fluids. Heat transfer fluids comprising these compounds can be used in all the mentioned applications involving heat exchange with an electronic computing equipment, batteries or semiconductor devices.

1. Fluorinated nitrocompounds having a general formula selected from (I) and (II): 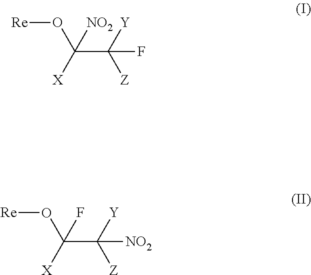
wherein
(i) X, Y and Z are independently selected from:
halogen,
hydrogen,
partially or fully halogenated C₁-C₄ alkyl,
with the proviso that at least one of X, Y or Z is a halogen, and
(ii) Re is selected from any C₁-C₇ linear, branched, saturated or unsaturated, or C₅-C₇ cyclic, saturated or unsaturated carbon chain which can comprise 0-5 heteroatoms selected from O and S, and can be, optionally, partially or fully halogenated;
with the proviso that if Re is CF₃ at least one of X, Y and Z is not F.

2. A method for exchanging heat with an object said method comprising using a heat transfer fluid wherein said heat transfer fluid comprises one or more fluorinated nitrocompounds having a general formula selected from (I) and (II): 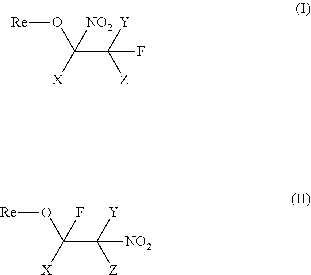
wherein
(i) X, Y and Z are independently selected from:
halogen,
hydrogen,
partially or fully halogenated C₁-C₄ alkyl,
with the proviso that at least one of X, Y or Z is a halogen, and
(ii) Re is selected from any C₁-C₇ linear, branched, saturated or unsaturated, or C₅-C₇ cyclic, saturated or unsaturated carbon chain which can comprise 0-5 heteroatoms selected from O and S and can be, optionally, partially or fully halogenated.

3. A method according to claim, 2 wherein said one or more fluorinated nitrocompounds having a general formula selected from (I) and (II) make up at least 5% by weight of said heat transfer fluid.

4. A method according to claim 2, wherein said heat transfer fluid has a GWP₁₀₀ determined according to the method reported in Hodnebrog et. Al. in Review of Geophysics, 51/2013, p 300-378, of less than 30, preferably less than 10, more preferably less than 5.

5. A method according to claim 2, wherein said object is an electronic computing equipment.

6. A method according to claim 5 wherein said electronic computing equipment is one or more servers.

7. A method according to claim 5, wherein said electronic computing equipment comprises one or more electronic circuit boards, said method comprising the step of contacting directly said electronic circuit boards with said heat transfer fluid.

8. A method according to claim 5, which is a method of single phase immersion cooling.

9. A method according to claim 5, which is a method of two phase immersion cooling.

10. A method according to claim 2, wherein said object is a battery, preferably a rechargeable battery.

11. A method according to claim 2, wherein said object is a semiconductor device.

12. A method according to claim 11 wherein one or more of a semiconductor processing equipment selected from an Etcher, an Asher, a Stepper and a plasma enhanced chemical vapour deposition (PECVD) chamber, is used, said semiconductor processing equipment including at least one temperature control unit (TCU) exchanging heat with said semiconductor device, wherein said TCU comprises said heat transfer fluid.

13. A method according to claim 11 wherein said method is a method for thermal shock testing of semiconductor devices, said method comprising, in any order:
i) cooling said semiconductor device to a temperature comprised from −10° C. and −100° C., using a first bath being made of a first fluid and
ii) heating said semiconductor to a temperature comprised from 60° C. and 250°, using a second bath being made of a second fluid,
wherein one of, or both, of said first and second fluids are heat transfer fluids according to claim 2.

14. A method according to claim 11 wherein said method is a method for vapor phase soldering of semiconductor devices, said method including
i) providing a semiconductor device comprising soldering paste,
ii) providing a closed chamber comprising said heat transfer fluid at its boiling point so that heated vapors of said heat transfer fluid are generated within said closed chamber, and
iii) introducing said semiconductor device in said closed chamber, in contact with said vapors of said heat transfer fluid thereby melting said soldering paste by contact with said heated vapors.

15. An apparatus comprising an electronic computing equipment and a heat transfer fluid wherein said heat transfer fluid comprises one or more fluorinated nitrocompounds having a general formula selected from (I) and (II): 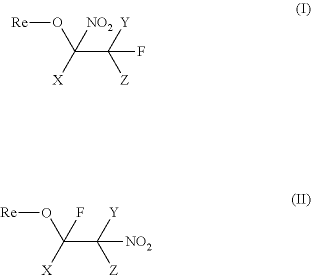
wherein
(i) X, Y and Z are independently selected from:
halogen,
hydrogen,
partially or fully halogenated C₁-C₄ alkyl,
with the proviso that at least one of X, Y or Z is a halogen, and
(ii) Re is selected from any C₁-C₇ linear, branched, saturated or unsaturated, or C₅-C₇ cyclic, saturated or unsaturated carbon chain which can comprise 0-5 heteroatoms selected from O and S and can be, optionally, partially or fully halogenated.

16. An apparatus comprising a battery, a thermal management system for said battery, said thermal management system for said battery comprising a heat transfer fluid exchanging heat with said battery, wherein said heat transfer fluid comprises one or more fluorinated nitrocompounds having a general formula selected from (I) and (II): 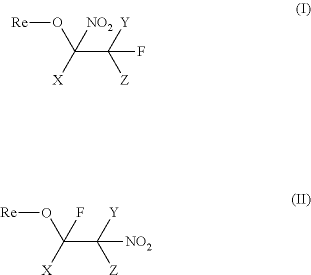
wherein
(i) X, Y and Z are independently selected from:
halogen,
hydrogen,
partially or fully halogenated C₁-C₄ alkyl,
with the proviso that at least one of X, Y or Z is a halogen, and
(ii) Re is selected from any C₁-C₇ linear, branched, saturated or unsaturated, or C₅-C₇ cyclic, saturated or unsaturated carbon chain which can comprise 0-5 heteroatoms selected from O and S and can be, optionally, partially or fully halogenated.
